(19) United States
(12) Patent Application Publication
(10) Pub. No.: US 2011/0153021 A1
(43) Pub. Date: Jun. 23, 2011

(54) NUCLEUS PULPOSUS REPLACEMENT DEVICE

(75) Inventor: Ashish Dhar DIWAN, Sydney (AU)

(73) Assignee: SPINECELL PTY LTD. ACN 114 462 725, Kogarah (AU)

(21) Appl. No.: 12/860,003

(22) Filed: Aug. 20, 2010

Related U.S. Application Data
(63) Continuation-in-part of application No. 10/530,152, filed on Jul. 1, 2005.

(30) Foreign Application Priority Data
Oct. 1, 2002 (AU) 2002951762

(51) Int. Cl. A61F 2/44

(52) U.S. Cl. 623/17.16

(57) ABSTRACT

A nucleus pulposus replacement device comprises a body of an elastomeric material which is able to be introduced and positioned within an annulus of an intervertebral disc of a patient. The material is of a form which undergoes a change from a first state, in which the body of material is able to conform substantially to a shape of a nuclear cavity of the intervertebral disc, to a second state, in which the body of material mimics bio-mechanical properties of a natural, healthy nucleus pulposus of an intervertebral disc. The material is of a consistency which inhibits leakage from an annulus fibrosis of the intervertebral disc.

### CROSS-REFERENCE TO RELATED APPLICATIONS

This application is a continuation-in-part application of U.S. Ser. No. 10/530,152 which was a national phase application of International Patent Application No. PCT/AU03/01289 having an international filing date of Sep. 30, 2003 and which claimed priority from Australian Provisional Patent Application No. 2002951762 dated Oct. 1, 2002. The contents of all the above applications are incorporated herein by reference in their entirety.

### TECHNICAL FIELD

The present invention relates to a system, device and method for imaging the interior of a bodily cavity of a patient; a system, device and method for mapping the interior of a bodily cavity of a patient; a method for implanting a nucleus pulposus replacement device, a delivery device for implanting a nucleus pulposus replacement device and a sealing device for sealing a bodily cavity of a patient.

### BACKGROUND ART

The human intervertebral disc (IVD) is a structure composed of a complex arrangement of various connective tissues. The structure of the IVD allows for its role in the effect of a functioning spinal column. Degeneration of the IVD is a consequence of aging and may begin as early as the first decade of life in males and the second decade in females. Disc degeneration plays a significant role in the aetiology of nucleus pulposus herniation, spinal stenosis and segmental spinal stability. Furthermore, IVD degeneration is implicated as a causative factor in mechanical lower back pain.

Over the years, there have been several suggestions and techniques relating to the development of prosthetic IVD replacement devices. Such devices include replacement of the entire intervertebral disc, and replacement of the nucleus pulposus only. Other methods of treatment include therapies for degenerated discs such as fusion and discectomy. Artificial devices are intended to restore or preserve the natural biomechanics of the intervertebral segment and to reduce further degeneration of adjacent levels of the spine.

Devices to replace the entire intervertebral disc include mechanical fixation devices which preserve the intersegmental stability using metallic end plates affixed to adjacent vertebra and an elastomeric rubber “nucleus” between the end plates. Other types of devices include “metal on metal” prostheses extending across adjacent vertebra.

Nucleus pulposus replacement devices involve substitution or augmentation of the nucleus pulposus in the event of IVD degeneration with normal annular architecture. Such devices include a prosthetic disc nucleus (eg. The PDN™ of RayMedica Inc., Minneapolis, Minn.), consisting of hyaluronic acid (hydroscopic gel) within a semi-permeable membrane that is enclosed in a woven jacket. A pair of these devices is inserted per level of the spine and, with time, an increased water content of the devices from absorption results in the volume of the devices expanding. Another such nucleus pulposus replacement device is the Aquarelle™ Hydrogel Disc Nucleus (Stryker Howmedica Osteonics, Rutherford, N.J.). This device consists of a hydrogel disc nucleus which is inserted, using instrumentation, into the intervertebral disc via a hole in the annulus, the hole having a cross-sectional area approximately one-quarter of that of the implant. The implant is composed of polyvinyl alcohol and water, its water content being high at intradiscal pressures found in the human lumbar spine. This property assists the implant to have a relatively low modulus of elasticity which allows it to conform to the vertebral end plates of the adjacent vertebrae.

The present inventor has identified shortcomings with the prior art and has developed a system which seeks to alleviate some of the shortcomings. The major shortcomings with the prior art include: —methods of implanting the nucleus replacement device that require a formal open approach with significant destruction of adjacent tissues including annulus; a lack of containment of implant material increasing the risk of leakage via annular fissures and tears; a lack of ability to recreate the kinematics of the disc motion segment and/or inability to bear load; risk of implant extrusion; bio-material of device being so novel that long term toxicity and performance are not established in humans.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### SUMMARY

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

It is to be noted that all aspects of described below are made possible by the ability to be performed percutaneously via a small stab incision in the skin using image guidance.

In a first aspect, a system for imaging the interior of a bodily cavity of a patient comprises:

a first imaging means able to be positioned within the bodily cavity and for producing a first image of the interior of the bodily cavity; and

at least a second imaging means able to be positioned within the bodily cavity and for producing a second image of the interior of the bodily cavity;

wherein the second imaging means is movable relative to the first imaging means and positionable in a location wherein the first image depicts the location of the second imaging means.

In an embodiment of the first aspect, the system may further comprise a display means for displaying the first and second images. The display means may comprise a first monitor for displaying the first image and at least a second monitor for displaying at least the second image. Instead, the display means may comprise one monitor that displays the first image and at least the second image. The system may further comprise an illuminating means for illuminating the cavity.

In another embodiment of the first aspect, the system may further comprise a tissue ablation means for ablating tissue in the bodily cavity, the ablation means being movable relative to the first imaging means. The first image may depict the location and orientation of the tissue ablation means. The tissue ablation means may be located adjacent to the second imaging means and the second image may depict the tissue undergoing ablation.

In a further embodiment of the first aspect, the tissue ablation means may be a radio-frequency ablation device or a plasma discharge device.

In yet another embodiment of the first aspect, the first imaging means may be a camera and the camera may be a video camera. The second imaging means may be a camera and may be a video camera. In each case, the camera can be an analogue or a digital camera.

In yet a further embodiment of the first aspect, the second imaging means may be an arthroscope. The arthroscope may include a flexible elongate portion having a camera positioned thereon that is insertable into the cavity, the flexible elongate portion allowing the portion of the periphery of the bodily cavity adjacent to the point of entry of the arthroscope to be viewed and accessed. The first imaging means and the second imaging means may be positioned on a support member and maintained in a spaced apart relationship relative to each other. The support member may be at least partially insertable into the bodily cavity. The first imaging means may be an arthroscope.

In still another embodiment of the first aspect, the system may further comprise

a position indication means able to be variably positioned within the bodily cavity;

a position detection means for receiving a signal from the position indication means; and

a processor means that analyses the signal and provides an output indicative of the location of the position indication means relative to the position detection means.

The signal may be selected from the group consisting of: infrared radiation, ultrasonic radiation, magnetic radiation, radio-frequency radiation, X-ray radiation and an optical image signal.

The position indication means may be a transmitter means and the position detection means may be a receiver means. Instead, the position indication means may be a reflector means and the position detection means may be a transceiver means. The signal may be firstly transmitted from the transceiver means and is then reflected by the reflector means back to the transceiver means.

The output of the processor means may be used to build a map of the bodily cavity. The system may further comprise a comparator display that displays a visual comparison of the map and a real image of the bodily cavity. The comparator display may allow determination of the orientation of the second imaging means in said cavity. The transmitter means may be able to be positioned at or adjacent the location of the second imaging means.

The real image may be obtained using an imaging technique selected from the group consisting of: X-ray imaging, magnetic resonance imaging, and computer tomography imaging. The real image may be obtained prior to mapping of the bodily cavity. Instead, the real image may be obtained during mapping of the bodily cavity. The real image may be continuously updated during mapping of the bodily cavity. The receiver means may be able to be positioned outside the bodily cavity. Instead, the receiver means may be able to be positioned within the bodily cavity.

The bodily cavity may be the nuclear space of an intervertebral disc or, instead, the bodily cavity may be a joint cavity.

In a second aspect, a system for mapping the interior of a bodily cavity of a patient comprises:

a position indication means able to be variably positioned within said bodily cavity;

a position detection means for receiving a signal from the position indication means; and

a processor means that analyses the signal and provides an output indicative of the location of the position indication means relative to the position detection means.

In an embodiment of the second aspect, the position indication means may be a transmitter means and the position detection means may be a receiver means. Instead, the position indication means may be a reflector means and the position detection means may be a transceiver means. The signal may be firstly transmitted from the transceiver means and may then be reflected by the reflector means back to the transceiver means.

The signal may be selected from the group consisting of: infrared radiation, ultrasonic radiation, magnetic radiation, radio-frequency radiation, X-ray radiation and an optical image signal.

In another embodiment of the second aspect, the output of the processor means may be used to build a map of the bodily cavity. The system may further comprise a comparator display that displays a visual comparison of the map and a real image of the bodily cavity. The real image may be obtained using an imaging technique selected from the group consisting of X-ray imaging, magnetic resonance imaging, and computer tomography imaging. The real image may be obtained prior to mapping of the bodily cavity. Instead, the real image may be obtained during mapping of the bodily cavity.

In a further embodiment of the second aspect, the system may further comprise a tissue ablation means for ablating tissue in the bodily cavity, the ablation means being movable relative to the position detection means and positioned adjacent to said position indication means such that the location of the position indication means is indicative of the location of the ablation means. The tissue ablation means may be a radio-frequency ablation device. Instead, the tissue ablation means may be a plasma discharge device. The real image may be continuously updated during the mapping of the bodily cavity.

In yet another embodiment of the present aspect, the position detection means may be able to be positioned outside the bodily cavity. Instead, the position detection means may be able to be positioned within the bodily cavity.

In yet a further embodiment of the second aspect, the system may further comprise a viewing means for imaging the interior of a bodily cavity of a patient, the viewing means comprising:

a first imaging means able to be positioned within the bodily cavity and for producing a first image of the interior of the bodily cavity; and

at least a second imaging means able to be positioned within the bodily cavity and for producing a second image of the interior of the bodily cavity;

wherein the second imaging means is movable relative to the first imaging means and able to be positioned in a location wherein the first image depicts the location of the second imaging means.

The bodily cavity may be the nuclear space of an intervertebral disc or a joint cavity.

In a third aspect, a method of imaging the interior of a bodily cavity of a patient comprises:

producing a first image of the interior of the bodily cavity wherein the first image is produced by a first imaging means able to be positioned within the interior of the bodily cavity;

producing at least a second image of the interior of the bodily cavity wherein the at least a second image is produced by a second imaging means able to be positioned within the interior of the bodily cavity; and

positioning the first imaging means in a location wherein the first image depicts the location of the second imaging means.

In an embodiment of the third aspect, the method may include the use of the system of the first aspect and associated embodiments.

In a fourth aspect, a method of mapping the interior of a bodily cavity of a patient comprises:

introducing a position indication means within the bodily cavity, the position indication means being able to be variably positioned within the bodily cavity;

positioning a position detection means to receive a signal from the position indication means; and

analysing the signal and providing an output indicative of the location of the position indication means relative to a position detection means.

The signal may be selected from the group consisting of: infrared radiation, ultrasonic radiation, magnetic radiation, radio-frequency radiation, X-ray radiation and an optical image signal.

In one embodiment of the fourth aspect, the analysing step may be performed by a processor means.

In another embodiment of the fourth aspect, the position indication means may be a transmitter means and the position detection means may be a receiver means. Instead, the position indication means may be a reflector means and the position detection means may be a transceiver means. The signal may be firstly transmitted from the transceiver means and may then be reflected by the reflector means back to the transceiver means.

In a further embodiment of the fourth aspect, the method may further comprise a step of using the output to build a map of the bodily cavity. The method may still further comprises a step of displaying the map of the bodily cavity on a display means. The method may further comprise a step of comparing the map with a real image of the bodily cavity.

The real image may be obtained using an imaging technique selected from the group consisting of: X-ray imaging, magnetic resonance imaging, and computer tomography imaging. The step of comparing the map with the real image may comprise:

determining the real position of the position detection means relative to the bodily cavity; and

superimposing the real position of the position detection means with the real image of the bodily cavity on the display means.

In yet another embodiment of the fourth aspect, the method may further comprise:

ablating at least a portion of the bodily cavity using an ablation means; and

updating the map during the ablation.

In yet a further embodiment of the fourth aspect, the method may include the use of the system of the third aspect and associated embodiments.

In a fifth aspect, a device for imaging the interior of a bodily cavity of a patient comprises:

a support member able to be at least partially positioned within the interior of the bodily cavity;

a first imaging means engageable with the support member for producing a first image of the interior of the bodily cavity; and

at least a second imaging means engageable with the support member for producing a second image of the interior of the bodily cavity;

wherein said second imaging means is movable relative to the first imaging means and able to be positioned at a location wherein the first image depicts the location of the second imaging means.

In an embodiment of the fifth aspect, the device may further comprise a tissue ablation means for ablating tissue in the bodily cavity, the ablation means being engageable with the support member and being moveable relative to the first imaging means. The tissue ablation means may be located adjacent to the second imaging means and the first image may depict the location and orientation of the tissue ablation means.

In another embodiment of the fifth aspect, the device may further include at least some of the embodiments of the first aspect.

In a sixth aspect, a device for mapping the interior of a bodily cavity of a patient comprises:

a support member able to be at least partially positioned within the bodily cavity;

a position indication means engageable with the support member and able to be variably positioned within the bodily cavity;

a position detection means for receiving a signal from the position indication means; and

a processor means that analyses the signal and provides an output indicative of the location of the position indication means relative to the position detection means.

The signal may be selected from the group consisting of: infrared radiation, ultrasonic radiation, magnetic radiation, radio-frequency radiation, X-ray radiation and an optical image signal.

In an embodiment of the sixth aspect, the position detection means may be engageable with the support member and may be able to be positioned within the bodily cavity.

In another embodiment of the sixth aspect, the position indication means may be a transmitter means and the position detection means may be a receiver means. Instead, the position indication means may be a reflector means and the position detection means may be a transceiver means. The signal may be firstly transmitted from the transceiver means and may then be reflected by the reflector means back to the transceiver means.

In a further embodiment of the sixth aspect, the system may further comprise a tissue ablation means for ablating tissue in the bodily cavity, the ablation means being engageable with the support member and being moveable relative to the position detection means. The tissue ablation means may be located adjacent to the position indication means.

In a seventh aspect, there is provided a nucleus pulposus replacement device which comprises a body of an elastomeric material which is able to be introduced and positioned within an annulus of an intervertebral disc of a patient, the material being of a form which undergoes a change from a first state, in which the body of material is able to conform substantially to a shape of a nuclear cavity of the intervertebral disc, to a second state, in which the body of material mimics bio-mechanical properties of a natural, healthy nucleus pulposus of an intervertebral disc and the material is of a consistency which inhibits leakage from an annulus fibrosis of the intervertebral disc.

Further, by conforming to the shape of the disc the device may be locked in between the central footprint of the vertebral bodies as the rim has a slight overhang which collectively will inhibit extrusion of the device.

At least in its second state, the body of material may substantially bear against and conform to internal boundaries of the annulus fibrosis of the intervertebral disc.

The body of material may have mechanical and visco-elastic properties suitable for structural support and load dampening in a spinal column of a patient. The material may also collectively restore the kinematics of the vertebral motion segment, consisting of the vertebra above and below with the interposed disc and facet joints, to a physiological state which in turn will help alleviate back pain in a patient and help inhibit further collapse and degeneration of the disc and related structures.

The device may comprise a membrane, or envelope, located about a periphery of the body of material to constrain the body of material within the nuclear cavity. The membrane may be substantially impermeable to the body of material. Further, the membrane may be flexible and may be non load-bearing.

The elastomeric material may be a silicone material. The material may be configured such that it cures after being implanted within the annulus of the intervertebral disc of the patient.

The device may include bioactive substances to be delivered to surrounding vertebral parts, such as the annulus of the intervertebral disc and end plates of adjacent vertebrae of the patient. The bioactive substances may be substances which induce cell growth and/or cell adhesion to the device. The adhesion of cells on the surfaces adjacent to the annulus may increase resistance to dislodgement of the device.

Further, the device may include drug delivery capabilities for at least one of active treatment and prophylactic treatment at a site of implantation of the body of material.

The device may include at least one of a radiopaque substance and a radiopaque marker for monitoring by X-ray during the operation and postoperatively. Examples of such radiopaque marking and monitoring materials include barium sulphate, zinc oxide, tantalum balls and iodine containing dyes.

The membrane may be modified to provide improved compressive stiffness. The membrane may be modified by having a side wall portion of greater thickness than surfaces of the membrane that abut end plates of adjacent vertebrae, in use. In addition, or instead, the membrane may be modified by being textured to have at least those surfaces of the membrane that abut end plates of adjacent vertebrae, in use, being of non-uniform thickness. The non-uniform thickness of the surfaces of the membrane may be provided by at least one of dimpling the surfaces and having studs protruding from the surfaces. Further the membrane may be modified by physical methods like plasma transformation, ionic or non-ionic transformation of the molecular structure of silicone so that the surface properties are rendered favourable for cell adhesion of either the annular fibroblasts, the vertebral endplate chondrocytes or the sub-chondral bone osteocytes.

The membrane may also be of an elastomeric material. Further, the membrane may be of the same material as the body of material so that, once the body of material has been injected into the membrane, a homogenous device results.

In an eighth aspect, there is provided a method of replacing the nucleus pulposus of an intervertebral disc of a patient using the device of the seventh aspect described above, the method comprising:

making an incision in an annulus fibrosis of the intervertebral disc;

introducing the body of material into vacated nuclear space of the intervertebral disc; and

allowing or causing the body of material to change from its first state to its second state such that it is constrained within the annulus of the intervertebral disc.

The incision may be made by stabbing and the incision may establish a working portal through which the body of material is introduced.

The method may include making the incision through the annulus fibrosis of the intervertebral disc via one of a posterior approach, a posterior-lateral approach, a lateral approach and an anterior approach to the disc.

The method may further include conducting a discectomy to form the vacated nuclear space. Optionally, the discectomy may be effected by ablation.

Further, the method may include distracting the intervertebral disc. The intervertebral disc may be distracted by way of an expansion means and/or by conventional traction. The intervertebral disc may be distracted by way of an expansion means passing through the incision in the annulus of the intervertebral disc and into the vacant nuclear space.

The expansion means may be a balloon device. The balloon device may be inflated by a fluid so as to distract the intervertebral disc. The balloon device may include radiopaque dye or markers which allow the position of the balloon to be monitored by an imaging means, such as X-ray, and facilitates pre-screening of disc placement. The fluid used to expand the balloon device may be biocompatible. Examples of suitable fluids include saline, PBS, iodine based dyes like those used in angiography and sterile water. In an embodiment, the method may include distracting the disc using the body of material.

The method may include irrigating the vacated nuclear space so as to remove any detritus such as debris, bone fragments and/or loose tissue.

After the intervertebral disc has been distracted by the balloon device, the method may include removing the balloon from the nuclear space. Further, the method may include, after distracting the disc, determining if there is any leak into the spinal column via the posterior annulus. This may be effected by injecting dilute barium sulphate-saline solution or a discography dye into the vacated nuclear space.

The method may include introducing the body of material into the vacated nuclear space of the intervertebral disc using a delivery device.

The method may include, initially, inserting a membrane into the vacated nuclear space and injecting the body of material into the membrane to be constrained by the membrane.

In a ninth aspect, there is provided the use of a silicone-based substance for the manufacture of a nucleus pulposus replacement device for the treatment of degenerative disc disease in the spine of a human being.

The nucleus pulposus replacement device can have one or more features according to the seventh aspect described above.

In a tenth aspect, a delivery system for implanting the device of the seventh aspect within an annulus of an intervertebral disc of a patient comprises:

a delivery device having a first end for the delivery of the body of material into the annulus whilst the material is in the first state; and

a release mechanism located at said first end of the delivery device for releasing the delivery device from the body of material following delivery of the device into the annulus.

The release mechanism may comprise a crimping means for disengaging the delivery device from the body of material when the material has changed into its second state.

The delivery device may further comprise a flow restrictor which allows the body of material to pass through the delivery device and through the release mechanism but which inhibits the material from flowing in the opposite direction and back into the delivery device.

The delivery device may further carry a non load-bearing expandable membrane. The membrane may be located adjacent the disengagement means and is able to be positioned about a periphery of the body of material. The membrane may be impermeable to the body of material and may remain about the body of material upon release of the body of material from the delivery device by the release mechanism.

In an eleventh aspect, an intervertebral disc distraction device comprises:

an elongate delivery member; and

an expansible distraction member carried by the delivery member.

The expansible distraction member may be an inflatable device, such as a balloon, that is expansible by a pressurised fluid so as to distract the intervertebral disc. The fluid used to expand the balloon may be biocompatible. Examples of suitable fluids include saline, PBS and sterile water. Instead, or in addition, the balloon may be expanded by a settable substance which changes from a first, fluent state to a second, set state, the settable substance being introduced into the balloon whilst in a less viscous first state and changes to a second more viscous state after expanding the balloon.

The expansible distraction member may comprise radiographic markers on its periphery for detection using radioopaque techniques. Instead, the expansible distraction member may be formed from a radiographic material.

The expansible distraction member may comprise an introduction portion, the introduction portion extending at least partially through the annulus of the intervertebral disc and the fluid may enter the balloon device through the introduction portion.

In a twelfth aspect, a sealing device for sealing a bodily cavity of a patient comprises:

an expansible membrane for insertion into the bodily cavity, the membrane comprising an envelope defining a chamber and having:
an internal surface;
an external surface; and
an aperture, said aperture providing a fluid pathway from the exterior of the membrane to the interior of said membrane, the introduction of a fluid through the aperture and into the interior of the membrane causes at least partial expansion of the membrane such that at least a part of the external surface comes into contact with at least a part of an internal periphery of the bodily cavity and, upon sealing of the aperture to retain the fluid within the interior of the membrane, the bodily cavity is sealed.

The membrane may further comprise radiographic marking means such that the location of the membrane is able to be monitored using imaging techniques.

The fluid may be at least partially settable and may be able to change from a first state to a second state, the second state having a viscosity greater than that of the first state.

The aperture of the membrane may be sealable by a sealing means, the sealing means being selected from the group consisting of: a valve, inherent properties of the material of the membrane, ultrasonic welding, temperature welding, UV light curing, sealant, clipping means and crimping

The expansible membrane may be compressible such that the sealing device can be inserted into the bodily cavity through an access aperture extending from an exterior of the cavity to an interior of the cavity.

The expansible membrane may further comprise an introduction portion through which the fluid is introduced into interior of the membrane, the introduction portion being in fluid communication with the aperture. The introduction portion may be formed integrally with the expansible membrane. The introduction portion may extend at least partially through the access aperture so as to provide a fluid pathway from an exterior of the bodily cavity to the interior of the membrane.

The bodily cavity may be vacated nuclear space of an intervertebral disc.

In a thirteenth aspect, a method of sealing a bodily cavity of a patient comprises:

inserting an expansible membrane into the bodily cavity, the membrane defining a chamber and having an access aperture;

expanding the expansible membrane by introducing a fluid into the chamber of the membrane through the aperture; and

closing the aperture to seal the chamber of the membrane to retain the fluid in the chamber of the membrane.

The fluid may be at least partially settable and may be able to change from a first state to a second state, the second state having a viscosity greater than that of the first state.

The aperture of the membrane may be closed using a sealing means, the sealing means being selected from the group consisting of: a valve, inherent properties of the material of the membrane, ultrasonic welding, temperature welding, UV light curing, sealant, clipping means and crimping.

The method may include the use of the sealing device of the twelfth aspect.

In a final aspect the entire system may be implanted along with a posterior dynamic stabilization system like the Diam device (Medtronic), X-Stop (Kyphon-Medtronics), a Wallis device (Abbott spine) or the likes or alternatively with a pedicle screw based posterior dynamic stabilization system like Dyneses (Zimmer), N-Flex (Synthes) or the DSS (Paradigm). This approach will provide for an anterior support for the posterior dynamisation system protecting the annulus, will replace the more disabling surgery of posterior spinal fusion by a dynamic motion preservation method of surgically managing back pain not responding to conservative treatment.

### BRIEF DESCRIPTION OF THE DRAWINGS

In the drawings:

FIG. 1 shows a superior-transverse view through an intervertebral disc of a patient;

FIG. 2 shows a schematic, anterior view of a disco-vertebral joint of a patient;

FIG. 3 shows a sectional, side view of a delivery device;

FIG. 4 shows a sectional, side view of a vertebral distraction device;

FIGS. 5(i) to 5(v) show steps in performing an annulotomy on, and distracting, an intervertebral disc of a patient;

FIGS. 6(i) to 6(iii) show superior-transverse views of implantation of a nucleus pulposus replacement device using a delivery device of FIG. 3;

FIG. 7 shows an example of a device for providing an interior map of the nuclear space of an intervertebral disc;

FIG. 8 shows a plan view of the use of the device of FIG. 7;

FIG. 9 shows a flow chart of a system for determining the geometry of the nuclear space of an intervertebral disc;

FIG. 10 shows a plan view of an embodiment of a nucleus pulposus replacement device partially inflated;

FIG. 11 shows a side view of the device of FIG. 10;

FIG. 12 shows a plan view of another embodiment of a nucleus pulposus replacement device;

FIG. 13 shows a side view of the device of FIG. 12; and

FIG. 14 shows a schematic, sectional side view of an embodiment of the device implanted between two verterbrae.

### DETAILED DESCRIPTION OF EXEMPLARY EMBODIMENTS

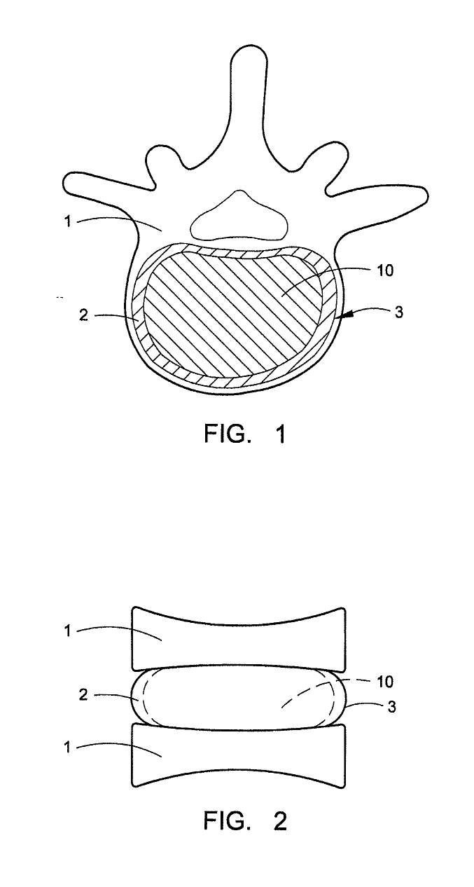

In FIG. 1 of the drawings, reference numeral 1 designates a vertebra. An intervertebral disc 3 is shown positioned relative to the vertebrae 1. The disc 3 comprises an annulus fibrosis, or annulus, 2 which surrounds a gelatinous nucleus pulposus, or nucleus, 10.

FIG. 2 shows the intervertebral disc 3 of the spine located between two adjacent vertebrae 1. The nucleus 10 of the intervertebral disc 3 is therefore bounded by the vertebrae 1 and the annulus 2.

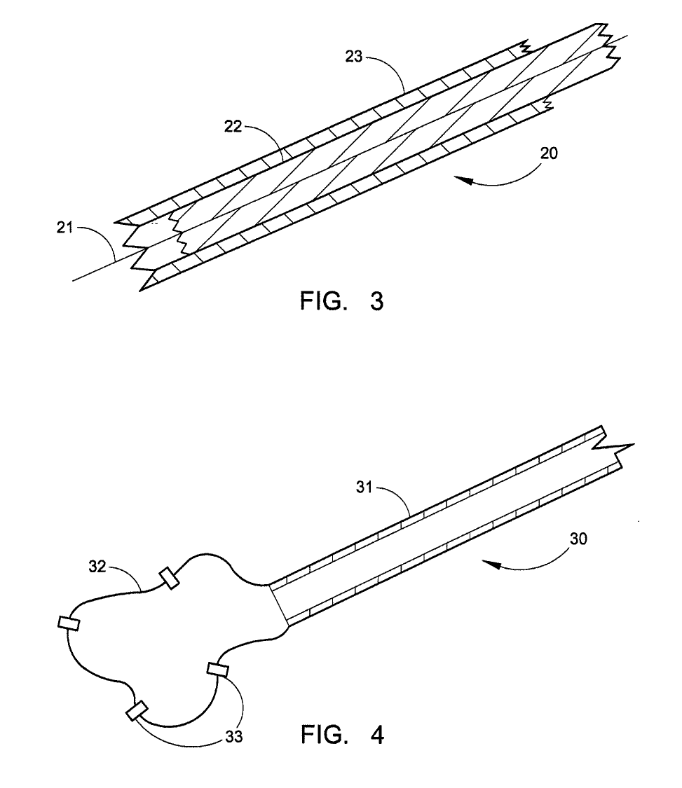

FIG. 3 is a sectional view of an annulotomy device 20 for performing an annulotomy on the annulus 2 of a degenerate intervertebral disc 3. The device 20 includes a localiser pin 21 concentrically positioned in a trocar member 22. An annulotomy member 23 is located about the trocar member 22. The localising pin 21, the trocar member 22 and the annulotomy member 23 are slidably arranged relative to one another.

The localiser pin 21 is formed of a biocompatible material, such as stainless steel, and has a diameter of about 1.5 mm. The trocar member 22 has a distal, internal diameter of about 1.55 mm such that the localiser member 21 can slide within the trocar member 22. The outer diameter of the trocar member 22 is preferably about 3.5 mm. The distal end of the trocar member 22 preferably has a serrated edge such that it can lock on to an outer surface of the annulus 2 of the intervertebral disc 3 with a significantly reduced likelihood of the trocar member 22 being dislodged from the annulus 22. The annulotomy member 23 also has a cutting edge at its distal end with an outer diameter of about 4.5 mm. The inner diameter of the annulotomy member 23 is slightly greater than the outer diameter of the trocar device such that sliding displacement between the trocar member 22 and the annulotomy member 23 is able to be achieved.

FIG. 4 of the drawings shows a distraction device 30. The distraction device 30 has an elongate, tubular delivery member 31 and an inflatable distraction member 32 mounted on a distal end of the delivery member 31. Preferably, the inflatable distraction member 32 is an inflatable balloon device that is inflatable by a pressurised liquid. Preferably, the liquid used is a bio-inert material including saline and physiological fluid.

A plurality of radio-opaque elements 33 are arranged on a periphery of the inflatable distraction member 32. The radio-opaque markers 33 are metallic or contain a metallic compound.

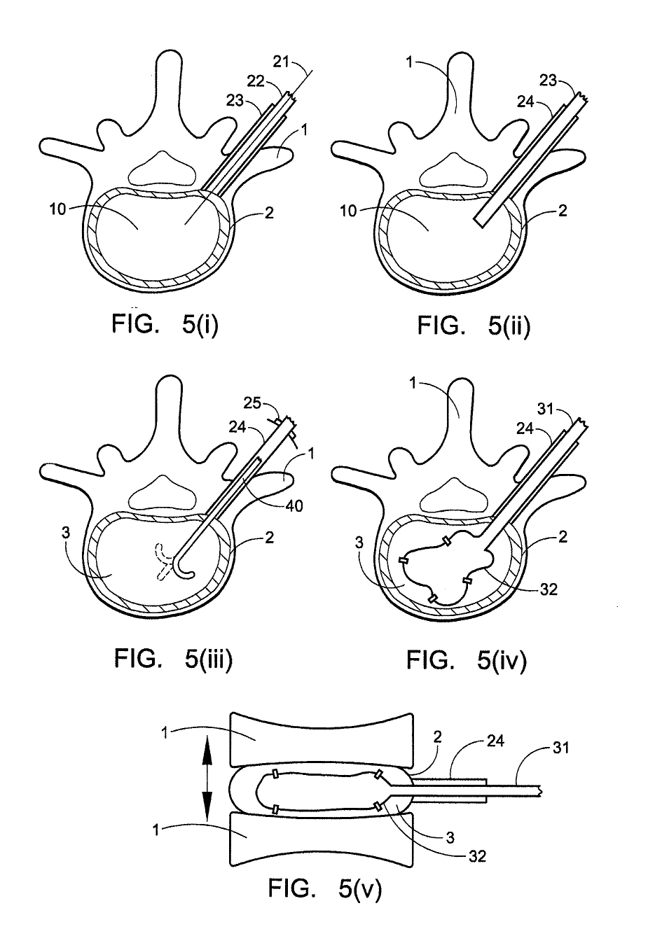

FIGS. 5(i) to 5(v) depict one example of the use of the annulotomy device 20 of FIG. 3 and the use of the intervertebral disc distraction device 30. FIG. 5(i) depicts how the annulotomy device 20 of FIG. 3 is placed, in use, in abutment with the outer surface of the annulus 2 of the intervertebral disc 3 using a posterio-lateral surgical approach. Other approaches can be utilised.

The serrated distal ends of each of the trocar member 22 and the annulotomy member 23 engage the outer surface of the annular wall 2 of the intervertebral disc 3. The localiser pin 21 is initially used to establish the position at which the annulotomy is to be performed. Once the localiser pin 21 is in position and the annulus 2 has been perforated by the localiser pin 21, the trocar member 22 and the annulotomy member 23 are guided to the outer surface of the annulus 2 along the localiser pin 21 until the distal ends of the trocar member 22 and the annulotomy member 23 are positioned at the outer surface of the annulus 2 of the intervertebral disc 3.

The annulotomy member 23 is then used to perforate the annulus 2 as shown in FIG. 5(ii) using the serrated cutting surface located at the distal end of the annulotomy member 23. The trocar member 22, by being engaged with the outer surface of the annulus 2, provides a support and acts as a guide for the annulotomy member 23 during the procedure.

A working cannula 24 having an inner diameter slightly greater than the outer diameter of the annulotomy member 23 is then positioned over the annulotomy member 23 using the annulotomy member 23 as a guide. A distal end of the cannula bears against the outer surface of the annulus 2 as shown in FIG. 5(ii). The working cannula 24 can have an engagement means for engaging the outer surface of the annulus 2. Such engagement means include pins, barbs, spikes, or the like.

The localiser pin 21 and the trocar member 22 are removed from the patient before or after the working cannula 24 is positioned relative to the annulus 2. Once the working cannula 24 has been placed in position, the annulotomy device 20 is withdrawn from the patient through the working cannula 24. A stabilisation device 25 is used externally of the patient to stabilise the working cannula 24 (see FIG. 5(iii)).

A nuclear material removal device 40 is then inserted through the working cannula as shown in FIG. 5(iii). It will be appreciated that, in the case of a degenerate disc 3, the nuclear material may have extruded out of the disc 3 and the use of the removal device 40 may not be necessary. The removal device 40 is used to remove nuclear material from the disc 3 to enable an implant to be inserted into a now vacant cavity of the intervertebral disc 3. The removal device 40 can be, for example, a mechanical device, such as a reaming tool or a rongeurs device, or a radio-frequency tissue ablation device.

Once nuclear material removal has been completed, the nuclear cavity is lavarged using saline or a physiological fluid. A radio-opaque die, for example, dilute barium sulphate solution is injected into the nuclear cavity and the cavity is scanned using radiographic techniques to determine the integrity of the annulus 2 and to determine if any leakage into the spinal canal of the patient has occurred. Arthroscopic techniques can also be employed through the working cannula 24 for inspection of the nuclear cavity.

The intervertebral space between the vertebrae 1 of the patient is distracted following removal of the nuclear tissue. Distraction is effected by traction and/or internal distraction using the distraction device 30 as shown in FIG. 4.

The material removal device 40 is withdrawn from the patient through the working cannula 24. The distraction member 32 of the distraction device 30 is inserted into the nuclear cavity through the working cannula 24, with the delivery member 31 extending through the working cannula 24 and out of the patient as shown in FIG. 5(iv).

Pressurised fluid, for example saline solution, is injected through the delivery member 31 and into the distraction member 32 to pressurise the nuclear cavity for a period of time such the distraction of the vertebrae 1 adjacent the intervertebral disc 3 occurs. The patient is imaged using radiographic techniques whilst the distraction member 32 is expanded so as to determine the geometric parameters of the nuclear cavity of the intervertebral disc 3, as shown in FIG. 5(v).

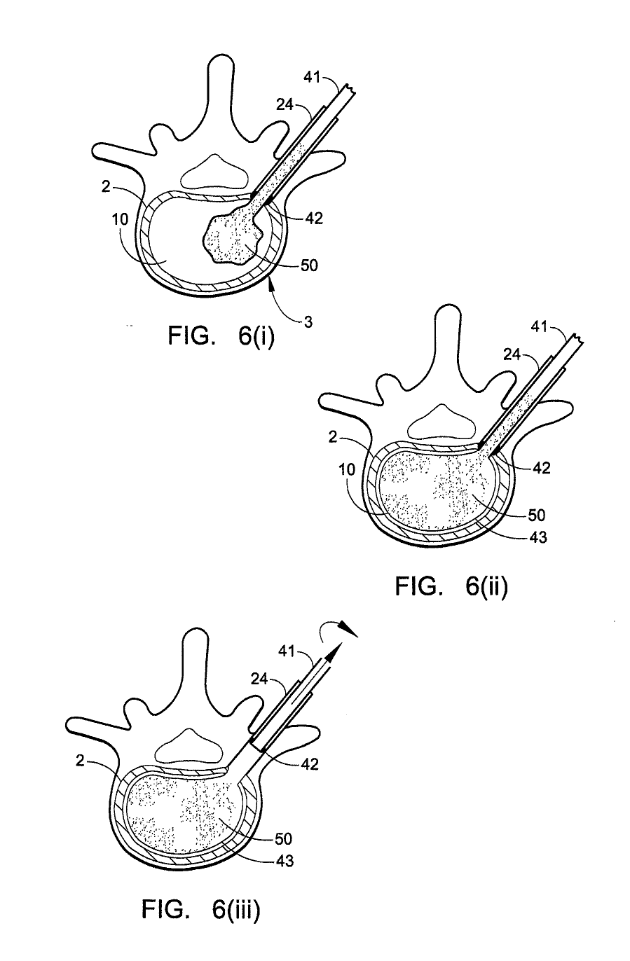

FIG. 6(i) depicts an embodiment of the implantation of a nucleus pulposus replacement device or implant within the nuclear cavity of the intervertebral disc 3. Implantation of the nucleus replacement implant follows the steps of the procedure as described above with reference to FIGS. 5(i) to 5(v).

A delivery device 41 is inserted through the working cannula 24 to the nuclear cavity of the intervertebral disc 3. The material 50 from which the nucleus replacement implant is to be formed is then injected through the delivery device 41 and into the nuclear cavity of the intervertebral disc 3, whilst the material 50 is in a first, fluent state suitable for injection.

The material 50 is then allowed to conform substantially to the interior of the nuclear cavity. The material 50 preferably has mechanical and visco-elastic properties suitable for nucleus replacement and which mimic the bio-mechanical properties of a natural nucleus of an intervertebral disc. An example of such a material 50 is a silicone-based material. Preferably, the material is self-curing by which the material changes to a second, set state having the required bio-mechanical properties of a natural nucleus of an intervertebral disc.

After curing of the material 50, a disengagement member 42 of the delivery device 41 allows the delivery device 41 to be disengaged from the cured material 50 and withdrawn through the working cannula 24. Remaining within the nucleus 10 is the nucleus replacement implant, formed of the cured material 50, substantially conforming to and constrained by the geometric boundaries of the annulus 2 and the vertebrae 1.

FIG. 6(ii) shows another embodiment of implantation of a nucleus replacement Oimplant, the implant including an outer membrane or envelope 43. During implantation, the envelope 43 is attached to a distal end of the delivery device 41 adjacent a distal end of the disengagement member 42. The disengagement member 42 is, for example, a push-off tube arranged co-axially with the delivery device 41.

The delivery device 41 is inserted into the working cannula 24 such that the envelope 43 is located within the nuclear cavity of the intervertebral disc 3. The material 50 which is to fill the envelope 43 is delivered in the same manner as described above with reference to FIG. 6(i). Upon injection and at least a degree of pressurisation, the filled envelope 43 substantially conforms to the volume of the nuclear cavity to form the implant. Upon curing, the delivery device 41 is disengaged from the implant, comprising the material 50 and the envelope 43, using the disengagement member 42 and is removed from the working cannula 24.

FIG. 6(iii) shows an example of removal of the delivery device 41, following the curing of the material 50 of the implant. In this example, the delivery device 41 is disengaged from the material 50 and the envelope 43 by rotating the delivery device 41 within the working cannula 24 and withdrawing the delivery device 41 through the working cannula 24.

The envelope 43 may be modified to promote conformance with the vacated nuclear space and to increase compressional stiffness. In one embodiment, sidewalls 86 (FIG. 14) of the envelope 43 are made thicker than surfaces 88 of the envelope 43 that abut the end plates of the vertebrae 1 after filling of the envelope 43 with the material 50. The sidewalls 86 of the envelope 43 abut an interior surface of the annulus 2 (not shown in FIG. 14).

In addition, or instead, at least an outer surface of the envelope 43 is modified by texturing the outer surface. In the embodiment of the envelope shown in FIGS. 10 and 11 of the drawings, an outer surface 80 of the envelope 43 is dimpled to increase surface area. This increases the coefficient of friction between the envelope 43 and surrounding vertebral parts such as the annulus 2 and the end plates of the adjacent vertebrae 1. An increased coefficient of friction results in increased compressive stiffness of the implant resulting in improved bio-mechanical properties of the implant.

In the embodiment of the envelope 43 shown in FIGS. 12 and 13 of the drawings, an outer surface 82 of the envelope 43 carries a plurality of spaced studs 84. These studs 84, once again, increase the surface area of the expanded envelope 43 in use resulting in a greater coefficient of friction and the resultant increased compressive stiffness of the implant.

Texturing the surface of the envelope 43 as described also has the benefit that filler-envelope interfacial stresses are reduced thereby reducing the likelihood of envelope-filler delamination. In addition, texturing minimises implant-tissue sliding. Still further, modifying the surface of the envelope 43 can be used, possibly in combination with bioactive agents, to initiate soft tissue attachment. Texturing of the outer surface of the envelope 43 also results in reduced third body wear occurring by housing debris remote from wear sites.

Texturing the outer surface of the envelope 43 as described above, results in a pattern of varying strain fields. The periodically varying nature of these strain fields also assists in regards to long term wear of the envelope 43. The varying strain patterns mitigate against the development of micro-fissures by diverting and arresting micro-fissures.

A further modification of the envelope 43 relates to its start-off geometry. By appropriate selection of the start-off geometry of the envelope 43, filling characteristics of the envelope 43 can be improved, i.e. the geometry of the envelope 43 is selected to conform most closely to the vacated nuclear space and to minimise unfilled spaces.

In certain embodiments, the filler material 50 is selected to have a Shore hardness of less than about 10 A, between 10 to 20 A, between 20 to 30 A, between 30 to 50 A, between 50 to 70 A or greater than 70 A, but preferably about 30 A. An example of a suitable filler material 50 is CSM-2186-14, manufactured by Nusil Technologies or MEDS-4230, manufactured by Nusil Technologies. In certain embodiments, the envelope 43 is made from liquid silicone rubbers. Examples include, but are not limited to, MED-4805, MED-4810, MED-4820, MED-4830, MED-4840 manufactured by Nusil Technologies. In certain embodiments, the envelope 43 is made from high consistency elastomers. Examples include, but are not limited to, MED-2174, MED4-4515, MED-4520, MED-4535 manufactured by Nusil Technologies. In certain embodiments, the envelope 43 is made from dispersions. Examples include, but are not limited to, MED-2214, MED-6400, MED-6600, MED1-6604, MED-6605 manufactured by Nusil Technologies.

In certain embodiments, the filler material 50 is a two-part pourable silicone elastomer, comprising Part A and Part B, that cures at room temperature. It contains about 5% BaSO4 (e.g., about 3%, 4%, 5%, 6%, or 7%) in both parts and mixes at a ratio of about 3:1 to 1:3 (e.g., 0.5:1 to 1.5:1, 1:1). The viscosity of Part A is about 105,000 cp (e.g., about 100,000 cp, 101,000 cp, 102,000 cp, 103,000 cp, 104,000 cp, 105,000 cp, 106,000 cp, 107,000 cp, 108,000 ep, 109,000 cp, or 110,000 cp) while the viscosity of Part B is about 71,000 cp (e.g., about 65,000 cp, 67,000 cp, 69,000 ep, 71,000 cp, 73000 cp, or 75,000 cp). Additionally, the filler material 50 may have a durometer of about 22-35 D2240 (e.g., about 22, 23, 24, 25, 26, 27, 28, 29, 30, 31, 32, 33 or 34), a tensile strength of between 850 to 1200 psi (e.g., about 900 psi, 950 psi, 1000 psi, 1050 psi, or 1100 psi), an elongation of between 500%-1200% (e.g., about 500%, 550%, 600%, 650%, 700%, 750%, 800%, 850%, 900% or 1000%), and a tear strength of between about 80-120 ppi (e.g., about 90 ppi, 95 ppi, 100 ppi, 105 ppi, or 110 ppi). The filler material 50 may typically be filled with inorganic material, for example silica, titanium dioxide, fly ash or other bio-acceptable fillers (e.g. amorphous silica). These fillers can optionally be surface treated with hydrophilic agents and/or hydrophobic agents. The inorganic fill material may be present in the filler material 50 in amounts between 5 and 50 wt. %, (e.g., 10-40 wt. % including 15 wt. %, 20 wt. % up to 30 wt. %, 35 wt. %, 40 wt. %). The inorganic fill material may be present in either Part A or Part B or both Parts A and B.

The material of the envelope 43 is typically a two-part translucent silicone system that cures rapidly with no required post-cure. It mixes at about a 3:1 to 1:3 ratio (e.g., 0.5:1 to 1.5:1 and 1:1). The composition may have a durometer of about 25-35 D2240 (e.g., about 26, 27, 28, 29, 30, 31, 32, 33, or 34), a tensile strength of between 1100 and 1500 psi (e.g., about 1250 psi, 1300 psi, 1350 psi, 1400 psi, or 1450 psi), an elongation of between 500% and 1100% (e.g., about 550%, 600%, 650%, 700%, 750%, 800%, 8505, 900%, 950% or 1000%), a tear strength of between 140 175 ppi (e.g., about 140 ppi, 145 ppi, 150 ppi, 155 ppi, or 160 ppi), and a stress at 200% strain of between 150 and 200 psi (e.g., about 160 psi, 165 psi, 170 psi, 175 psi, 180 psi, 185 psi, or 190 psi).

In embodiments, the silicone used for the filler material 50 or for the material of the envelope 43 may include any one of a variety of silicones generally referred to as bio-compatible elastomers formed from polysiloxanes or polyorganosiloxanes which are polymers having the general chemical formula [R2SiO]n, where R is any suitable organic group and n is any integer. Such polysiloxanes suitable for these purposes may also include a broad family of more complex synthetic polymers containing a repeating silicon-oxygen backbone with organic side groups attached via carbon-silicon bonds. Such complex silicones, or polymeric siloxanes, may be linear, branched or cross-linked, and can be represented by the formula [RpSiO(4−p/2)]m, where p is 1-3, m>1, and R is any suitable organic group such as alkyl, alkenyl, fluoroalkyl, phenyl, vinyl, hydroxyl, alkoxy, amino or alkylamino or combination of one or more of these organic groups, e.g., -phenylvinyl. The term silicone as used herein is also meant to include elastomers that are hetero- or copolymers of the above-described polysiloxanes. The polysiloxanes suitable for the present invention may also have their terminal ends such as alkyl, alkenyl, fluoroalkyl, phenyl, hydride, vinyl, hydroxyl, alkoxy, amino or alkylamino group or combinations of one or more of these organic groups, e.g., -alkylvinyl (this could be Part A of a two-part system). The polysiloxanes suitable for example as a counterpart polysiloxane (e.g., Part B) can be modified to include functional, active or inactive organic groups for various purposes, such as to promote crosslinking (for example hydrides or other terminal groups functional groups suitable for treating with ethylenically unsaturated functional groups) or for copolymerization or other reactions. The two groups undergo an addition reaction during curing. Such addition reaction can be aided by a Group VIII metal (e.g., platinum, rhodium, or palladium).

Non-limiting examples of some polysiloxanes include: polydiorganosiloxanes, polyaklysiloxanes, polydialkylsiloxanes, polydimethylsiloxanes, polyaminoalyklsitoxanes, polyaminoalklsiloxanes, polyethyleneglycol-polydimethyl siloxane co-polymers, silicone polyesters, polysiloxane-polylactone copolymers, polydimethyldiphenylsiloxane, polyalkylsiloxane-polyurethane copolymers with one or more terminal groups such as alkyl, alkenyl, fluoroalkyl, phenyl, vinyl, hydroxyl, alkoxy, amino or alkylamino group or combination of two, three or more of these groups (e.g., -alkylvinyl). In certain embodiments, the envelope 43 is made from a silicone rubber material having the following characteristics:
a Shore hardness (A scale) in the range from about 20-50;
a tensile strength in the range from about 2700 kPa to 11000 kPa;
an elongation of between about 400% and 800%; and
a tear strength of between about 1700 kg/m and 4500 kg/m.

The filler material 50 is also of a silicone rubber material which, prior to use, is stored in two separate parts. The filler material 50, comprising the combined parts, when mixed in a ratio of 1:1 and cured, has the following characteristics:
a Shore hardness (A scale) in the range from about 20 to 40, more particularly, about 25 to 30 and, optimally, about 28;
a tensile strength in the range form about 7000 kPa to about 9500 kPa, more particularly, about 8000 kPa to about 9000 kPa and, optimally, about 8500 kPa;
an elongation in the range from about 550% to 700%, more particularly, about 600% to 650% and optimally, about 640%; and
a tear strength in the range from about 1000 to 2000 kg/m, more particularly, about 1250 kg/m to 1750 kg/m and, optimally, about 1500 kg/m.

One example of a suitable material for the filler material 50 has the following characteristics after mixing the parts in a 1:1 ratio and after curing:
a Shore hardness (A scale) of 28;
a tensile strength of 8439 kPa;
an elongation of 639%; and
a tear strength of 1500 kg/m.

The filler material 50 may be treated to contain 5%, by volume, barium sulphate to appear radio-opaque under X-ray, CT, fluoroscopy and MRI. In addition, the filler material 50 contains a catalyst and has a scorch time of between about 1.5 to 2.5 minutes with a curing time of about 5 minutes. When the filler material 50 is charged into the envelope 43 it causes inflation or expansion of the envelope 43 in an elastically deformable manner. Expansion of the envelope 43 can occur to such an extent that, where necessary, the expanded envelope 43 distracts the adjacent vertebrae 1 to restore the original spacing between the vertebrae 1. By using radio-opacity in the filler material 50, distraction of the vertebrae 1 can be monitored in real time using a fluoroscope or the similar equipment.

Further, the envelope 43 conforms to the shape of the vacated nuclear cavity. Because the envelope 43 expands within the cavity and conforms closely to the shape of the cavity, the envelope 43 self anchors within the cavity and “extrusion” of a unified nucleus pulposus replacement device, comprising the envelope 43 and the filler material 50, formed through the aperture previously formed in the annulus 2 of the disc 3 is inhibited.

The material for the envelope 43 may, depending on the grade or class of material used, be post cured for a period of time. This is effected by placing the moulded envelope 43 into an oven, for example, for a period of about 1 to 4 hours at a temperature of about 150° C. to 180° C.

By having the material of the envelope 43 and the filler material 50 of the same type, but different grades or classes, chemical bonding between the materials is enhanced which encourages the formation of the nucleus pulposus replacement device.

An embodiment of the biomaterial was studied to characterize the mechanical and wear behaviour of the device (also referred to below as an “implant”).

Fatigue testing was performed to evaluate the mechanical and wear performance of the implant over its intended life. Fatigue testing in compression, flexion/extension, lateral bending and axial rotation were conducted to mimic in vivo physiological ranges. Specimens were loaded to 10 million cycles in compression as suggested by ASTM 2346-05 and 5 million cycles in flexion/extension, lateral bending and axial rotation.

The test implant was an annulus model (Silicone Shore Hardness 60A) with a complete implant (filler material—CSM-2186-14 (Nusil Technologies) and envelope material—MED-4830 (Nusil Technologies) and Calf Serum 30 g/L solution (as per ISO/DIS 18192-1)) injected according to expected surgical procedure. Six implants were created.

The annulus model was placed between two Perspex constraining plates which prevent the model from bulging superiorly and inferiorly. Through the annulotomy, the implant was delivered using the equipment described herein until the implant had completely filled the cavity of the annulus model. The annulus model and the implant were placed inside a water bath set to 37° C. and left to cure for at least 1 hour.

The six specimen implants were glued to the test platens and left to dry for 24 hours. The specimens and test platens were then connected to the spinesimulator. The test stain was filled with calf serum and maintained at 37±3° C.

The test execution was as follows: —

1) A compression load of 100 N and 600 N was applied and the heights of the specimens at these loads were measured. This height was taken as the reference heights

2) The specimens were cyclically loaded under the following conditions:—
Compression
   Load range:
      600 N to 2000 N for 10 000 cycles
      600 N to 1500 N for 990 000 cycles
   Load frequency: 2 Hz
Flexion/Extension
   Bending range: +6/−3°
   Range frequency: 1 Hz
Lateral Bending
   Bending Range: ±2°
   Range frequency: 1 Hz
Axial Rotation
   Bending Range: ±2°
   Range frequency: 1 Hz

3) After the completion of the 1 million compression cycles a 100 N and 600 N load was reapplied to measure the height change.

4) This process was repeated another 9 times such that the specimens underwent 10 million compression cycles.

5) At the completion of the cycling loading the specimens were left to recover for 24 hours and then the 100 N and 600 N loads were reapplied to measure the height change.

After each million compression cycles the calf serum test medium was collected and analyzed Since literature publications have suggested the standing load results in approximately 0.5 MPa of pressure in the lumbar discs while disc pressures whilst lifting is suggested to be between 1.0 to 2.3 MPa, it was believed that choosing a loading regime between 600 N to 1500 N and 600 N to 2000 N would represent a worse case scenario. The flexion/extension, lateral bending and axial rotations ranges are comparable to human in vivo conditions as suggested by ISO/DIS18192-1. The frequency of 2 Hz was chosen so as to not overheat the specimens.

In the fatigue test, one of the six specimens was destroyed due to it slipping from the stainless steel platen at about the 5.8 million cycle mark. Tears in the annulus were noticed in all test stations at the 3 million cycle mark.

Observations of the implant were graded to the scale below.
Grade 1=Jacket peeling observed
Grade 2=Minor cracks observed
Grade 3=Progression of minor cracks observed
Grade 4=Major crack
   Wear particles collected in the test medium were subjected to SEM (Scanning Electron Microscope). The results characterized the size with respect to shape factor, roundness and equivalent circle diameter. The test medium was collected every million cycles and wear particles extracted. The number of particles found per million cycles was collated. The number of particles found per sample per million cycles ranged from 137 to 797 particles. The average number of particles per million cycles was approximately 500 particles. Most particles had a shape factor of between 0.9 and 1 indicating that most of the particles collected were round. The equivalent circle diameter for most particles was between 0.1 and 0.3 μm.

EDX (Energy dispersive X-ray spectroscopy) analysis of the wear particles showed no trace of barium, while silicon, gold and palladium were detected. The detection of gold and palladium was due to contamination via the SEM analysis. A sample of an untested implant was also analyzed under EDX to determine the detectability of barium. The analysis showed barium was detected but the wear particles collected from the fatigue testing did not show any signs of barium. According to the supplier of the composition, the barium sulfate particles contained within the filler material is approximately 1 μm. Hence the EDX analysis is sensitive enough to detect the presence of barium sulfate particles, but the lack of traces detected by the EDX for the implant indicated that the implant had not worn or that the wear had not been significant enough.

All 5 remaining specimens passed the acceptance criteria which required the specimens to not split up into more than 3 distinct pieces which are smaller than the size of the annulotomy. This criterion was chosen as the mechanical function of the implant will remain even if it has broken up so long as the implant is adequately constrained within the annulus. So long as the implant is able to maintain its total volume it will still function as required. The 5 specimens all remained intact in one piece when the annulus remains essentially intact. In the tests involving Specimens 2 and 4, it was noted that the simulated annulus failed leading to a grading of higher than 1 for these tests at some point beyond 5 million cycles. It is noted that a protocol involving the replacement of the annulus after a set number of cycles, e.g., 2 million, may more closely represent the natural regeneration of the annulus that occurs in the body and provide a better measure of the performance of the implant. In spite of these shortcomings in the simulated annulus, the structural integrity of the 5 specimens remained intact after the fatigue testing and hence the acceptance criteria were met. The EDX analysis on the wear particles generated from the testing procedure showed no signs of barium or platinum and hence not from the nucleus filler material.

The acceptance criteria also required no more than 10% of the volume lost. From visual observation of the 5 specimens, there were no sites where significant parts of the implants were worn away. Specimens tested where the annulus model did not fail remained fully intact with no cracks. The remaining two specimens where the annulus failed had cracks present in them but nonetheless remained intact as one functional body. Accordingly, the implant is capable of withstanding in vivo conditions for 10 years equivalent with supra-physiological loading.

Supra physiological loads in the lumbar spine may be encountered during accidents. Thus evaluation of the impact performance of the implant is required.

The test set up for shock testing was as follows:

1) Specimens were loaded in compression to 100 N to measure the reference height.

2) A shock load of 3000 N at a rate of 200 kN/min was then applied.

3) Specimens were then unloaded to 100 N at a rate of 200 kN/min and held for 20 seconds to measure the reference height.

This particular test was performed because a shock load rate of 250 mm/min or greater has been suggested by ASTM draft standard WK4863.

| | Permanent |
|---|---|
| Specimen | deformation (mm) |
| 2.1 | 0.5 |
| 2.2 | 0.5 |
| 2.3 | 0.4 |
| 2.4 | 0.3 |
| 2.5 | 0.3 |
| 2.6 | 0.4 |
| Mean | 0.45 |
| Std. dev. | 0.09 |

The mean permanent height loss for the specimens was 0.45 mm or 3.2%. The permanent deformation of the implant constrained within an annulus model is less than 4%.

In vivo, lumbar discs encounter both static and dynamic loading. Conducting static testing is essential in understanding the creep and recovery behaviour of the implant under a constant load.

1) Specimens were loaded in compression to 100 N to measure the reference height and then unloaded.

2) Specimens were loaded in compression to 600 N and held continuously for 16 hours.

3) A load of 100 N was applied to measure the height following static creep.

4) Specimen was unloaded for 8 hours for recovery.

5) A 100 N load was reapplied to measure the recovery and permanent deformation from that measured in step 1.

6) Steps 1 to 5 were repeated.

This test was performed because a 600 N load over 16 hours is approximately equivalent to a person standing continuously for 16 hours. The loading regime of the specimens aimed to simulate a person standing continuously for two 16 hour periods followed by 8 hours of rest over 48 hour period. At the first 600 N compression load all specimens crept less than 0.2 mm over the 16 hour period which is equivalent to less than 1.5% height loss. At the second 600 N load all test specimens crept less that 0.2 mm, again equivalent to less than 1.5% height loss.

The specimens were also subjected to a 100 N reference height load before the commencement of testing. The 100 N load was also applied before and after the 8 hour no load (rest periods). In average height loss at 100 N load at the end of testing was 0.2 mm when compared to the reference height. The maximum height loss at 100 N load occurred after the second 600 N loading period and it showed the height loss at this load was approximately 0.3 mm when compared to the reference height.

This indicates the implant loses minimal height after constant static loading. The static creep of the implant constrained within an artificial annulus model creeps less than 2% over a 16 hour period.

Other nucleus replacement prostheses, mainly hydrogels, require fluid absorption to form the required dimensional characteristics and thus swelling tests are essential in the mechanical characterization process. The implant is not made from a hydro-expanding material. It allows water molecules to pass through, therefore this test was not considered necessary. It was included for completeness and to verify the above claim.

Specimens were dried in an oven at temperatures above 100 degrees for a minimum of 4 hours.

1) Specimens were placed within a swell test jig with a plastic plate placed on top.

2) The jig was then filled with Ringer's solution.

3) A LVDT transducer was used to measure the height change over a 48 hour period.

| | Max. sensor | Min. sensor | Fluctuation | Height Change |
|---|---|---|---|---|
| | deflection | deflection | Range | after 48 hours |
| Specimen | (mm) | (mm) | (mm) | (mm) |
| 1 | 0.02 | −0.02 | 0.04 | −0.01 |
| 2 | 0.01 | −0.01 | 0.02 | 0.01 |
| 3 | 0.01 | 0.00 | 0.01 | 0.01 |
| 4 | 0.00 | −0.02 | 0.02 | −0.01 |
| 5 | 0.00 | −0.03 | 0.03 | −0.02 |
| 6 | 0.00 | −0.02 | 0.02 | −0.01 |
| Mean | 0.01 | −0.02 | 0.02 | −0.01 |
| Std. dev. | 0.01 | 0.01 | 0.01 | 0.01 |

The results show the mean height change after 48 hours soaking in Ringer's Solution was 0 mm. The maximum change in height occurred on specimen 5 with a 0.03 mm. The results indicate that the implant is not affected by swelling through fluid absorption as opposed to hydrogels.

Previous clinical studies of other prostheses have raised concern with extrusion of the device. Therefore, it was important to evaluate the risk of extrusion with the implant. The proposed surgical procedure used to implant the device is through the creation of an annulotomy. Therefore this extrusion test was done on a similar sized annulotomy in an artificial annulus model (this being the worst case opening in the annulus). Because of the characteristics of the implant, it does not really lend itself to extrusion. This test was performed for completeness and no extrusion of any kind or severity was expected.

The implant was partially filled to a volume between 1.5 to 2 ml inside the annulus cavity to represent a worst case scenario since it was believed that partially filled implants have a greater chance of extrusion due to their relative size to the annulotomy opening.

1) Specimens were fatigue loaded for 200,000 compression cycles under the following conditions:
Compression
   Load range: 600 N to 2000 N
   Frequency: 2 Hz
Flexion/Extension
   +6/−3° Frequency: 1 Hz
   Frequency: 1 Hz

Partially filled implants (30 to 50% fill) were subjected to fatigue testing in compression and flexion/extension. The position of the annulotomy was positioned such that the annulotomy underwent tension during the flexion cycle. During this cycle the implant and the encompassing annulus model were flexed to 6 degrees. This, accompanied with the compression cycles, subjected the implant to conditions that would induce expulsion. After 200,000 cycles no expulsions or protrusions were observed in any of the test specimens. Detachment between the superior section of the annulus and the stainless steel test platen occurred in specimens 3 and 4 after the 200,000 cycles.

A partially filled implant (30 to 50% fill) was chosen as a smaller sample would more likely extrude than a fully filled implant as the size of the annulotomy remained the same. Also the implant was inflated through the annulotomy and hence the proximal end of the implant sits at the inner edge of the annulotomy. In addition to this test, no expulsions were observed during the fatigue test in which the implant was subjected to multi-directional testing to 10 years equivalent with an annulotomy present. From the literature expulsion studies have been conducted using cadaveric models. This test was performed in an artificial annulus model as it would allow testing to be conducted to 200,000 cycles which would otherwise not be possible in a cadaveric test model due to tissue degeneration.

No expulsions or protrusions were observed for all 6 test articles after 200,000 cycles hence the acceptance criteria were met. In addition no expulsions were observed during any point of the fatigue test.

Due to the viscoelastic nature of the implant, it was expected to creep under an applied load. The following test aimed to evaluate creep. An implant specimen was filled into a 25.4 mm diameter cylindrical mould to approximately 10.5 mm in height.

1) The specimen was placed between delrin platens

2) The specimen was then subjected to a 253 N (0.5 MPa) compression load for 16 hours.

3) The specimen was then unloaded (no load applied) for 8 hours to recovery.

4) Steps 2 and 3 were repeated a further three times such that the specimen was subjected to four 16 hour loading regimes over a four day per period.

| Time Point | Height Loss (%) |
|---|---|
| End of first session | −3.47 |
| Start of 2^{nd} session | −0.71 |
| End of 2^{nd} session | −4.18 |
| Start of 3^{rd} session | −1.10 |
| End of 3^{rd} session | −4.44 |
| Start of 4^{th} session | −1.69 |
| End of 4^{th} session | −4.53 |

The results show a gradual decrease in height during the loading periods (approximately 3.5% per 16 hour period). During the 8 hour rest periods the specimen recovered approximately 80% of the height loss. During loading on the fourth day aspects of recovery were observed. The implant showed signs of permanent deformation and recovery after loading due to its viscoelastic properties.

Conducting mechanical tests on aged samples is critical in ensuring the mechanical performance of the implant is not compromised over time. Specimen implants were aged using a 10 degree temperature acceleration method suggested by the literature. All specimens were subjected to 11 hours in a dry oven at 177° C. and then placed in a saline water bath for 46 days at 87° C. This subjected the specimens to 24 years equivalent worth of aging. It has been suggested that an increase of 10° C. doubles the aging process. Therefore, subjecting the samples to the above heating conditions was equivalent to at least 24 years worth of aging.

The specimens were glued to the test platens and left to dry for 24 hours. The specimens and test platens were then connected to the spinesimulator. The test stain was filled with calf serum and maintained at 37±3° C.

The test execution was as follows: —

1) A compression load of 100 N and 600 N was applied and the heights of the specimens at these loads were measured. These heights were taken as the reference heights.

2) Specimens were cyclically loaded under the following conditions:—
Compression
   Load range:
      600 N to 2000 N for 10 000 cycles
      600 N to 1500 N for 990 000 cycles
      Load frequency: 2 Hz
Flexion/Extension
   Bending range: +6/−3°
   Range frequency: 1 Hz
Lateral Bending
   Bending Range: ±2°
   Range frequency: 1 Hz
Axial Rotation
   Bending Range: ±2°
   Range frequency: 1 Hz

3) After completion of the 1 million compression cycles a 100 N load and a 600 N load were reapplied to measure the height change.

All specimens were loaded to 100 N and 600 N and the heights measured at this load. After the specimens were subjected to cyclic load the 100 N and 600 N load was reapplied to measure the heights. These values were compared to the reference heights.

| | Height loss at 100N | Height loss at 600N |
|---|---|---|
| Specimen | reference load | reference load |
| 3.1 | 0.53 | 1.4 |
| 3.2 | 0.49 | 1.3 |
| 3.3 | 0.44 | 1.3 |
| 3.4 | 0.45 | 1.1 |
| 3.5 | 0.55 | 1.3 |
| 3.6 | 0.46 | 1.2 |
| Mean | 0.49 | 1.3 |
| Std. dev. | 0.1 | 0.1 |

The average height loss at the 100 N and 600 N reference loads was 0.49 mm and 1.3 mm, respectively. The height measurements after 1 million cycles showed the aged specimens performed better than the fatigue specimens in terms of height loss.

No cracks were observed on any of the specimens and aging does not have any serious adverse mechanical effects on the implant.

Height maintenance is an important mechanical function in a nucleus replacement device. The following test aimed to evaluate the dynamic fatigue properties of the implant constrained within an artificial annulus model.

The filler material 50 (CSM-2186-14) was injected into the annulus cavity via a 4 mm annulotomy and left to cure for 24 hours.

1) The specimens were placed between the two delrin platens (see FIG. 10.1)

2) The specimens were subjected to a 509 N compressive load to reduce creep effects.

3) The specimens were then subjected to a cyclic compression loading between 509 N and 1730 N at 2 Hz for 100,000 cycles.

The change in peak height during the cyclic loading and the change in height during the cyclic loading were measured.

The maximum and minimum height (at 509 N and 1730 N load respectively) of the specimens were recorded for the predetermined cycles. A reduction in height during the 1 million cycles (dynamic creep) was evident in both specimens where the greatest observable difference was recorded between cycles 1 and 5,000. The rate of height loss (dynamic creep) plateaus out between cycles 5,000 to 100,000.

Cycling the specimens between 509 N (0.5 MPa) and 1730 N (1.7 MPa) is approximately equivalent to a person standing in a relaxed position to and lifting a 20 kg load. Cycling the specimens in this fashion is thus a gross over-exaggeration of what a person would encounter in everyday life. However the aim was to test the lifecycle of the device in a worst case scenario at accelerated loading conditions and was thus felt to be justified.

The dynamic creep of the implant constrained within an annulus model over 100,000 cycles was less than 5%.

A finite element analysis of the implant was also performed, and the following items were observed from the model.

The implant is believed to restore the nucleotomy model to near-physiological axial displacement when the implant completely fills the vacated nuclear space. Data indicates that the implant axial displacement approaches the result provided by the intact model. In contrast to this, the untreated nucleotomy results in an abnormally low axial stiffness.

The extent of the nucleotomy relative to the nucleus volume does not have as pronounced an effect on the axial stiffness when compared to the extent the implant fills the nucleotomy. This is apparent when the implant model (based on a finite element analysis) (100% filling of nucleotomy) is compared with a partial implant. The partial implants and new inflation models (30%, 70%) do not show significant difference between each other. This phenomenon relies on the assumption that a void remains between the implant and the nucleotomy in the partial-fill implant.

The use of materials like silicones are well suited for a nucleus pulposus replacement application because it is a viscoelastic material which means it is capable of providing the shock absorbing requirements of the motion segment. Under a given load, the prosthesis formed of the silicone material deforms and is capable of distributing the applied load radially to evenly distribute the load across the endplates of the vertebrae and to the annulus. This reduces the risk of the implant subsiding into the endplates and restores the intradiscal pressure which restores the hoop stresses to the annulus. More importantly, the nucleus prosthesis is elastically deformable. Thus, the application of force causes the nucleus prosthesis to deform elastically so that, once the force has been removed, the prosthesis will return to its relaxed, undeformed state.

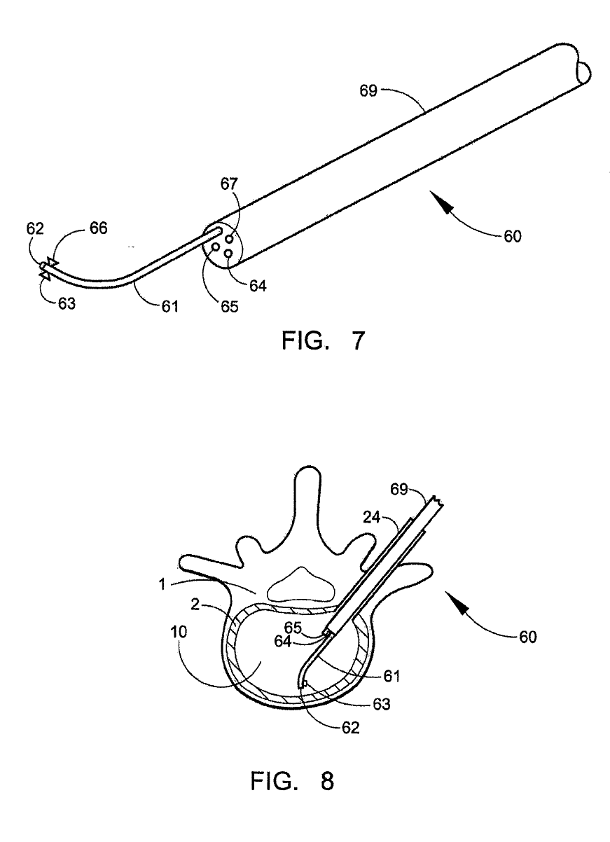

FIG. 7 depicts an example of a device 60 that is used to generate an interior map of the nuclear cavity of an intervertebral disc 3 of a patient. The device 60 includes a transmitter 63 and a receiver 64. The transmitter 63 is located at, or in proximity to, the distal end of a flexible portion 61 of the device 60. The position and orientation of the flexible portion 61 is controllable by the surgeon from a position externally of the body of the patient, such that the position of the transmitter 63 is variable relative to the position of the receiver 64.

The transmitter 63 transmits a signal to the receiver 64 that allows determination of the position of the transmitter 63 relative to the receiver 64. An example of a suitable transmission medium is infra-red. In this example, the transmitter 63 is in direct line-of-sight from the receiver 64. Instead, a reflector may be positioned at the distal end of the flexible portion and the transmitter 63 located adjacent the receiver 64, or be integral with the receiver in the form of a transceiver.

The device 60 further includes a first camera 62 located at the distal end of the flexible portion 61, and a second camera 65. A support member 69 maintains the first camera 62 and the second camera 65 in a spaced apart relationship relative to each other such that an image provided by the second camera 65 depicts the location of the first camera 62.

The first camera 62 and/or the second camera 65 may be a video camera. A digital image obtained by the second camera 65 provides for position tracking of the first camera 62 by image analysis techniques. The second camera 65 may be an arthroscope and the flexible portion 61 may be a portion of the arthroscope. A light source 67 is also included for illumination to allow imaging by the camera 62 in the visible light spectrum.

A nuclear material removal device, such as an ablation device, 66 is also located at the distal end of the flexible portion 61. Examples of suitable ablation devices 66 include a radio-frequency type probe, a plasma discharge device, or the like.

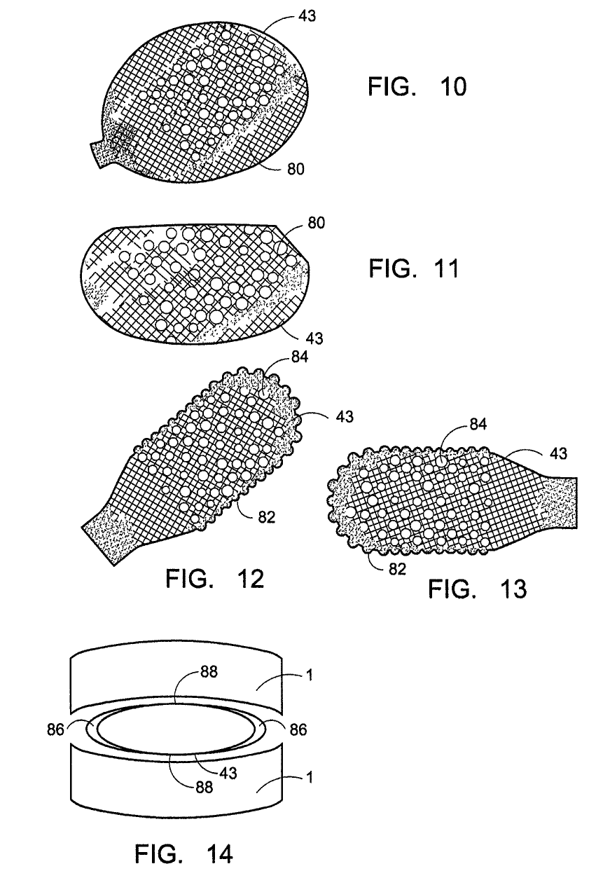

FIG. 8 depicts an example of the use of the device 60 of FIG. 7. The device 60 is used for ablating the nucleus 10 of the intervertebral disc 3 and mapping the periphery of the vacated nuclear cavity. The device 60 is at least partially inserted within the nuclear space of the intervertebral disc 3 through the working cannula 24 after performance of the annulotomy described above so that a distal end of the device 60 abuts the nuclear material of the nucleus 10 of the disc 3. Examples of suitable surgical approaches include posterio-lateral approach and an anterior approach.

The first camera 62 is located at the distal end of the flexible portion 61. The ablation device 66 is used to ablate the nucleus 10 of the disc 3. The region at which ablation occurs is imaged by the camera 62 and so provides an output visible to the surgeon during the procedure. The second camera 65 allows for overall imaging of the distal end of the device 60 and the visual monitoring of the ablation device 66 during ablation assists in ensuring appropriate use of the ablation device 66 during the surgical procedure.

The transmitter 63, located at the distal end of the flexible portion 61 outputs a signal indicative of the location of the distal end of the device 60 relative to the receiver 64 and hence the location within the nuclear cavity. In this example of the device 60, the receiver 64 is also located within the nuclear cavity, although it will be appreciated that the receiver 64 could be located externally of the body of the patient. Examples of suitable modes of transmission of the signal in the present example are infra-red transmission and radio-frequency transmission.

The position of the transmitter 63 relative to the receiver 64 can be processed by an external processor so as to allow generation of an internal map of the nucleus 10. Transmission of the signal from the transmitter 63 is continuous, intermittent or user-determined.

The user positions the distal end of the device 60 at a position within the nucleus 10, with the aid of the second camera 65 and externally operates the transmitter 63 so as to determine the coordinates or position of the transmitter 63. Multiple transmissions at various locations along the periphery of the nuclear cavity allow development of a map or visual representation that is indicative of the volume and geometry of the nuclear cavity.

The map or visual representation of the nuclear cavity output by the processor is compared with real, pre-obtained or simultaneously obtained images of the nucleus from various imaging techniques, such as X-ray, computer aided tomography, ultrasound and magnetic resonance imaging. Further to this, the image may be overlayed with the map of the nucleus to allow ready determination of the degree of ablation required and/or monitoring of the position of the device 60.

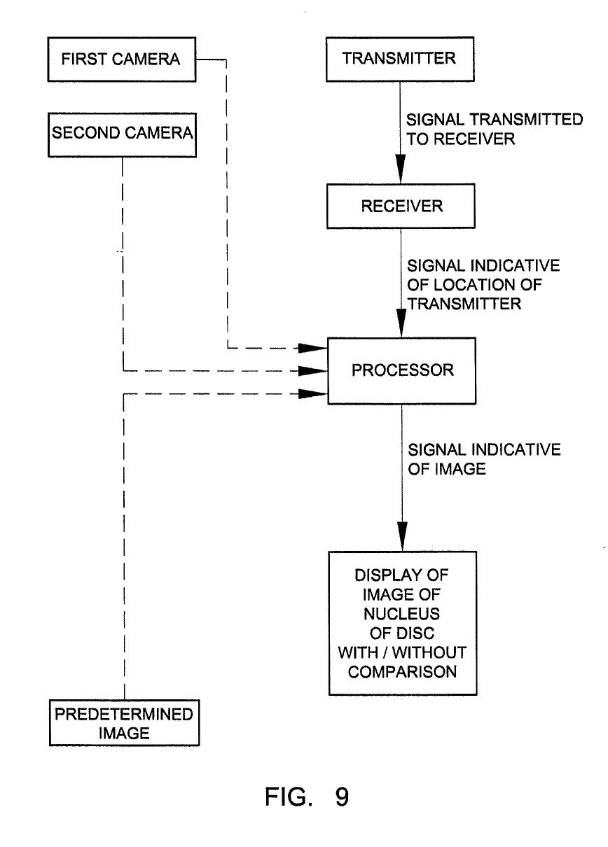

FIG. 9 is a flow chart of a representative system that uses the data of device 60. The system shown in FIG. 9 also provides visual monitoring of the ablation device 66 by the second camera 65 and visual monitoring of the portion of the nucleus being ablated and assessment of tissue by the first camera 62. Visual monitoring is provided by a first monitor for display of an image from the first camera 62 and a second monitor for display of an image from the second camera 65. Alternatively, a single monitor can display the images from both the first camera 62 and the second camera 65.

The image provided by the processor is displayed on a comparator display with the internal map provided by the processor as described with reference to FIG. 8 with the real image in real time. As tissue is ablated by the ablation device, the map can be updated and compared with the real image. Such an updating of the image allows a user to determine the new real image of the cavity being mapped and allow a user to know where within the cavity the ablation device 66 is located, by way of superimposition of the updated image with the predetermined real image.

The comparator display is incorporated with the display which displays the image from the first camera 62 and the image from the second camera 65. It will be appreciated that the receiver may be located within or outside of the bodily cavity and that any bodily cavity of a patient may be mapped in this way, including the interior nuclear space of an intervertebral disc of a patient.

A system incorporating such features enables a surgeon to assess the interior space of an intervertebral disc of a patient and to be provided with information as to where a surgical instrument is located within the intervertebral disc. Furthermore, data indicative of the internal geometry of the intervertebral disc of a patient provided by such a system allows selection of an appropriately sized implant for nuclear pulposus replacement.

It will be appreciated by persons skilled in the art that numerous variations and/or modifications may be made to the described embodiments without departing from the scope of the appended claims. The present embodiments are, therefore, to be considered in all respects as illustrative and not restrictive.

1. A nucleus pulposus replacement device which comprises a body of an elastomeric material which is able to be introduced and positioned within an annulus of an intervertebral disc of a patient, the material being of a form which undergoes a change from a first state, in which the body of material is able to conform substantially to a shape of a nuclear cavity of the intervertebral disc, to a second state, in which the body of material mimics bio-mechanical properties of a natural, healthy nucleus pulposus of an intervertebral disc and the material is of a consistency which inhibits leakage from an annulus fibrosis of the intervertebral disc.

2. The device of claim 1 which comprises a membrane located about a periphery of the body of material to constrain the body of material within the nuclear cavity.

3. The device of claim 2 in which the membrane is substantially impermeable to the body of material.

4. The device of claim 2 in which the membrane is flexible and is non load-bearing.

5. The device of claim 1 in which the elastomeric material is a silicone material.

6. The device of claim 1 which includes bioactive substances to be delivered to surrounding vertebral parts.

7. The device of claim 1 which includes drug delivery capabilities for at least one of active treatment and prophylactic treatment at a site of implantation of the body of material.

8. The device of claim 1 which includes at least one of a radioactive substance and a radiopaque marker.

9. The device of claim 2 in which the membrane is modified to provide improved compressive stiffness.

10. The device of claim 9 in which the membrane is modified by having a side wall portion of greater thickness than surfaces of the membrane that abut end plates of adjacent vertebrae, in use.

11. The device of claim 9 in which the membrane is modified by being textured to have at least those surfaces of the membrane that abut end plates of adjacent vertebrae, in use, being of non-uniform thickness.

12. The device of claim 10 in which the non-uniform thickness of the surfaces of the membrane is provided by at least one of dimpling the surfaces and having studs protruding from the surfaces.

13. The device of claim 2 in which the membrane is of an elastomeric material.

14. The device of claim 2 in which the membrane is of the same material as the body of material so that, once the body of material has been injected into the membrane, a homogenous device results.

15. A method of replacing the nucleus pulposus of an intervertebral disc of a patient using the device of claim 1, the method comprising:
making an incision in an annulus fibrosis of the intervertebral disc;
introducing the body of material into vacated nuclear space of the intervertebral disc; and
allowing or causing the body of material to change from its first state to its second state such that it is constrained within the annulus of the intervertebral disc.

16. The method of claim 15 which includes making the incision through the annulus fibrosis of the intervertebral disc via one of a posterior approach, a lateral approach, a posterior-lateral approach and an anterior approach to the disc.

17. The method of claim 15 which includes conducting a discectomy to form the vacated nuclear space.

18. The method of claim 15 which includes distracting the intervertebral disc.

19. The method of claim 17 which includes distracting the disc using the body of material.

20. The method of claim 15 which includes irrigating the vacated nuclear space so as to remove any detritus.

21. The method of claim 18 which includes, after distracting the disc, determining if there is any leak into the spinal column.

22. The method of claim 15 which includes introducing the body of material into the vacated nuclear space of the intervertebral disc using a delivery device.

23. The method of claim 15 which includes, initially, inserting a membrane into the vacated nuclear space and injecting the body of material into the membrane to be constrained by the membrane.

24. The use of a silicone-based substance for the manufacture of a nucleus pulposus replacement device for the treatment of degenerative disc disease in the spine of a human being.
